(19) United States
(12) Patent Application Publication
(10) Pub. No.: US 2011/0168803 A1
(43) Pub. Date: Jul. 14, 2011

(54) MICROCRYSTALLINE WAX CAPSULE PERFUME DIFFUSING DEVICE

(76) Inventor: Huang Liwei, Shenzhen (CN)

(21) Appl. No.: 12/850,900

(22) Filed: Aug. 5, 2010

(30) Foreign Application Priority Data
Jan. 12, 2010 (CN) 201020026522.6

(51) Int. Cl. A61L 9/04

(52) U.S. Cl. 239/60

(57) ABSTRACT

The invention relates to perfume diffusing devices, particularly, to a microcrystalline wax housing perfume diffusing device, comprising a light-transmitting microcrystalline wax housing and electronic component. The housing has a first cavity zone inside which perfumed volatile material, being perfume-volatilizable, is placed. The electronic component is inside the housing; and includes a LED light, battery compartment, and power switch. The perfumed volatile material in the first cavity zone can volatilize perfume, and the light from the LED of the electronic component can penetrate the housing and shine out, so the device functions as ornamentation and illumination, which richens the function of the device and widens its applications. The housing is formed of microcrystalline wax and can emit perfume itself, hence the invention has a relatively long performance life because when the perfumed volatile material is used up or unable to emit perfume, the microcrystalline wax can still emit perfume itself.

This application is based on and claims the benefit of priority from Chinese Patent Application No. 201020026522.6, filed on Jan. 12, 2010, the contents of which are incorporated herein by reference.

### TECHNICAL FIELD

The present invention relates to the technical field of perfume diffusing devices, more particularly to a microcrystalline wax housing perfume diffusing device.

### BACKGROUND

The prior perfume diffusing devices generally include a housing, being formed of ceramics, glass, polymer, acrylic, etc., and perfumed volatile material is placed in the housing. The perfumed volatile material emits perfume so that the smell of the surrounding air is changed. However, these perfume diffusing devices only offer a function of volatilizing perfume or ornamentation. Hence, the single function of the prior perfume diffusing devices has limited their application. Furthermore, the housing of the prior perfume diffusing devices can not emit perfume themselves, and do not offer the function of emitting perfume when the perfumed volatile material is used up or otherwise unable to emit perfume. As a result, the prior perfume diffusing devices have relatively short performance life.

### SUMMARY OF THE INVENTION

Considering the deficiency of the prior art, the objective of the present invention is to provide a microcrystalline wax housing perfume diffusing device which offers functions of ornamentation and illumination in addition to perfume diffusing.

To achieve the aforementioned objective, the technical solution used in the present invention is as follows:

A microcrystalline wax housing perfume diffusing device comprising a light-transmitting microcrystalline wax housing and an electronic component, wherein the microcrystalline wax housing has a first cavity zone, inside which perfumed volatile material, being perfume-volatilizable, is placed; wherein the electronic component is installed inside the microcrystalline wax housing, the electronic component includes a LED light, a battery compartment and a power switch herein.

A second cavity zone for installing the electronic component is arranged on the bottom of the microcrystalline wax housing, the first cavity zone is located above the second cavity zone, and a hole for placing the LED light is positioned between the first cavity zone and the second cavity zone.

The electronic component has at least one LED light.

The perfumed volatile material is a scent.

The perfumed volatile material may also be a concentrated scent wax particles.

The perfumed volatile material may also be a natural dried flower.

The beneficial advantages of the present invention include:

The present invention provides a microcrystalline wax housing perfume diffusing device including: a light-transmitting microcrystalline wax housing and an electronic component, wherein the microcrystalline wax housing has a first cavity zone, inside which perfumed volatile material, being perfume-volatilizable, is placed; wherein the electronic component is installed in the microcrystalline wax housing, and the electronic component includes an LED light, a battery compartment and a power switch therein. In use, the perfumed volatile material placed in the first cavity zone of the present invention can volatilize perfume, and the light from the LED of the electronic component can penetrate the microcrystal and shine out, so that the present invention offers additional functions of ornamentation and illumination, which richens the function of the perfume diffusing device and widens its application. Furthermore, the microcrystalline wax housing of the present invention is formed of microcrystalline wax and can emit perfume itself, hence the present invention has a relatively long performance life because when the perfumed volatile material is used up or otherwise unable to emit perfume, the microcrystalline wax can still emit perfume itself.

### DESCRIPTION OF THE DRAWINGS

FIG. 1 is a schematic structural view of the present invention from top perspective.

FIG. 2 is a schematic structural view of the present invention from bottom perspective.

FIG. 3 is a cross-section view of the present invention.

FIG. 4 is a cross-section view of a microcrystalline wax housing of the present invention.

FIG. 5 is an enlarged partial sectional view of the microcrystalline wax housing, which shows the inner and outer housing of the microcrystalline wax housing and an LED in between.

FIG. 6 shows one arrangement of the LEDs disposed around the microcrystalline wax housing.

### PREFERRED EMBODIMENTS

The present invention will be further described in combination with the accompanying drawings.

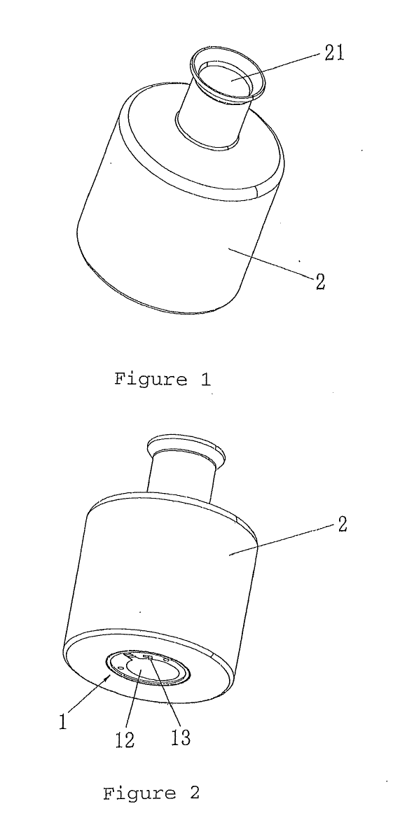

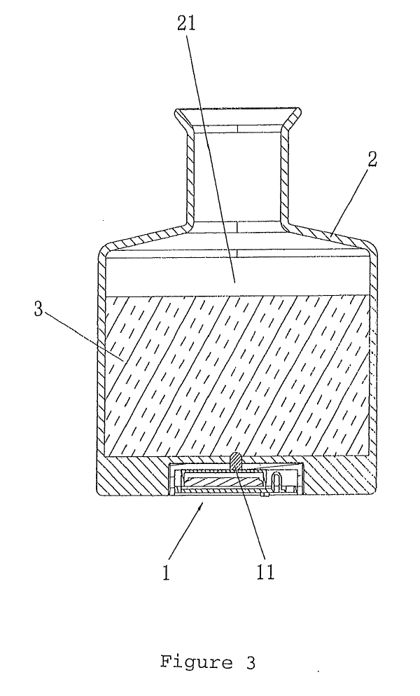

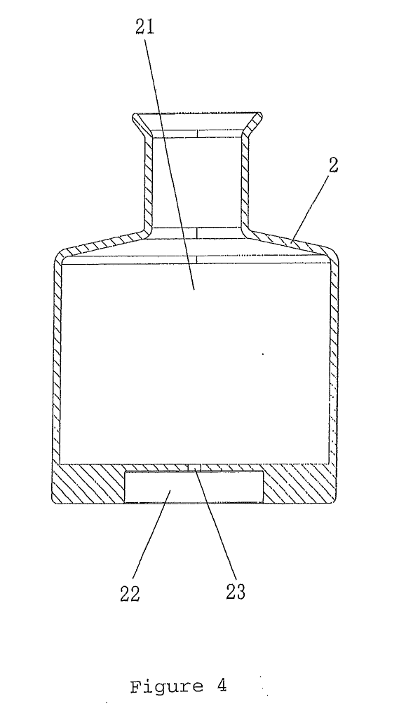

Referring to FIGS. 1-6, the present invention includes a light-transmitting microcrystalline wax housing 2 and an electronic component 1, wherein, the microcrystalline wax housing 2 has a first cavity zone 21 and a perfumed volatile material 3 is placed in the first cavity zone 21; the electronic component 1 is installed within the microcrystalline wax housing 2, the electronic component 1 includes an LED 11, a battery compartment 12 and a power switch 13. When a proper battery is placed into the battery compartment 12 and the power switch 13 is on, the LED 11 can be powered and the light from the LED 11 can shine out through the microcrystalline wax housing 2, so that the present invention offers functions of ornamentation and illumination. A perfume volatile material is added to the synthetic material of the microcrystalline wax housing 2 of the present invention, as result of which, the microcrystalline wax housing 2 can emit perfume. The present invention has a relatively long performance life because when the perfumed volatile material 3 is used up or otherwise unable to emit perfume, the microcrystalline wax 2 can still emit perfume itself.

In the present embodiment, said perfumed volatile material 3 is a scent, said perfumed volatile material may also, of course, be perfume diffusing materials such as a concentrated scent wax particle, a natural dry flower, etc. If said perfumed volatile material 3 is the scent, a volatilization stick may be inserted into the first cavity zone 21, in other words, one end of the volatilization stick is inserted into the scent, and the other end of the volatilization stick extends out of an opening of the first cavity zone 21, so that the scent in the first cavity zone 21 can volatilize through the volatilization stick.

A second cavity zone 22 for installing the electronic component 1 is positioned on the bottom of the microcrystalline wax housing of the present embodiment, a first cavity zone 21 is located above the second cavity zone 22, and a hole 23 for placing a LED light 11 is positioned between the first cavity zone 21 and the second cavity zone 22 so that the light from the LED 11 can shine out through the microcrystalline wax housing 2.

The electronic component 1 of the present embodiment has at least one LED 11. Of course, a plurality of LEDs 11 may be installed onto the electronic component 1 of the present embodiment; in the present embodiment, a compact cell such as a lithium cell etc. is placed into a battery compartment 12 to provide power to the LED 11. It should be understood that, in the present embodiment, other types of battery may also be used as power supply according to product requirements.

In the present embodiment, said microcrystalline wax housing 2 is formed of light-transmitting microcrystalline wax, so that the light from the LED 11 can shine out through the microcrystalline wax housing 2. Of course, in addition to bottle-shaped, said microcrystalline wax housing 2 of the present embodiment can also be Christmas tree-shaped, Santa Claus-shaped, engraved or embossed, snowman-shaped, etc. according to different ornamental occasions, as long as the chosen shape provides good decorative effect. Furthermore, to enhance the decorative effect, an ornament, such as an imitated person, an imitated animal, an imitated architecture, etc., may be placed onto the outer surface of said microcrystalline wax housing 2, or different shapes and colors of decorative decals may be applied onto the outer surface of said microcrystalline wax housing 2. The microcrystalline wax housing 2 of the present invention can withstand high-temperature (72˜80° C.). Furthermore, compared to housings made of glass, etc, the microcrystalline wax housing 2 of the present invention is more shatterproof, for example, when falling from the height of about 80˜120 cm.

In summary, the present invention has perfume diffusing function as general perfume diffusing devices, furthermore, the LED 11 positioned on the electronic component 1 of the present invention has decorative and illuminating functions, so that the functions of the perfume diffusing are enriched and its application is widened.

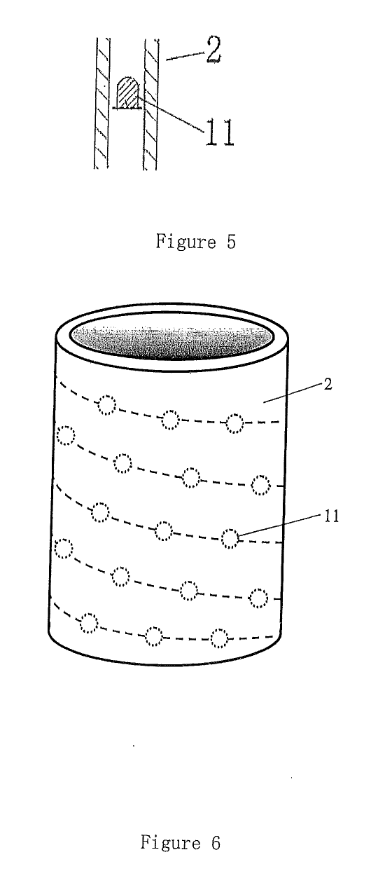

In one preferred embodiment, a plurality of LEDs 11 is provided and arranged around the microcrystalline wax housing 2 so as to display a specific or various patterns. It is understandable that various manners can be adopted to arrange the LEDs around the microcrystalline wax housing 2. For example, to facilitate the installation of the LEDs 11, the microcrystalline wax housing 2 can comprise an inner housing and an outer housing, as shown in FIG. 5. The LEDs 11 can be installed between the inner and outer housings as shown in FIG. 5. Alternatively, the LEDs 11 (as well as the connection wires) can be embedded in the microcrystalline wax housing 2 during the formation of the microcrystalline wax housing 2. In this case, it is not necessary to provide both inner and outer housings.

FIG. 6 shows one arrangement of a plurality of LEDs 11 positioned in the microcrystalline wax housing 2. The LEDs 11 are positioned heliacally around the microcrystalline wax housing 2. One skilled in the art would understand that the arrangement of FIG. 6 just shows a specific arrangement pattern. Various patterns would be possible if desired.

Of course, above-mentioned is only for some of the preferred embodiments of the present invention. Hence, equivalent variations and modifications based on the configurations, features, and principles described in the present patent application should be in the scope of the present invention.

What is claimed is:

1. A microcrystalline wax housing perfume diffusing device comprising:
a light-transmitting microcrystalline wax housing (2) having a first cavity zone (21) where a perfumed volatile material (3), being perfume-volatilizable, is placed, the microcrystalline wax housing (2) containing a perfumed material which enables the microcrystalline wax housing itself to emit perfume.

2. The microcrystalline wax housing perfume diffusing device of claim 1, wherein a second cavity zone (22) for installing an electronic component (1) is positioned on the bottom of said microcrystalline wax housing (2), the first cavity zone (21) is located above the second cavity zone (22), and a hole (23) for placing a LED (11) is positioned between the first cavity zone (21) and the second cavity zone (22).

3. The microcrystalline wax housing perfume diffusing device of claim 2, wherein said electronic component (1) has at least one LED (11).

4. The microcrystalline wax housing perfume diffusing device of claim 3, wherein said perfumed volatile material (3) is a scent.

5. The microcrystalline wax housing perfume diffusing device of claim 3, wherein said perfumed volatile material (3) is a concentrated scent wax particle.

6. The microcrystalline wax housing perfume diffusing device of claim 3, wherein said perfumed volatile material (3) is a natural dried flower.

7. The microcrystalline wax housing perfume diffusing device of claim 1, wherein an electronic component (1) is installed inside the microcrystalline wax housing (2), the electronic component (1) has an LED (11), a battery compartment (12) and a power switch (13).

8. The microcrystalline wax housing perfume diffusing device of claim 1, wherein said microcrystalline wax housing is capable of withstanding high-temperature of 72˜80° C.

9. The microcrystalline wax housing perfume diffusing device of claim 2, wherein the LED extends beyond the second cavity zone into the first cavity zone.

10. The microcrystalline wax housing perfume diffusing device of claim 1, wherein a plurality of LEDs are arranged around the microcrystalline wax housing (2).

11. The microcrystalline wax housing perfume diffusing device of claim 10, wherein the plurality of LEDs are embedded in the microcrystalline wax housing.

12. The microcrystalline wax housing perfume diffusing device of claim 11, wherein the plurality of LEDs are positioned heliacally around the microcrystalline wax housing.

13. A microcrystalline wax housing perfume diffusing device comprising
a light-transmitting microcrystalline wax housing (2), the microcrystalline wax housing (2) having a first cavity zone (21) in which a perfumed volatile material (3), being perfume-volatilizable, is placed, and having a second cavity zone (22) for installing an electronic component (1), the first cavity zone (21) being located above the second cavity zone (22), a hole (23) for placing an LED (11) being positioned between the first cavity zone (21) and the second cavity zone (22).

14. A microcrystalline wax housing perfume diffusing device of claim 13, wherein said electronic component (1) has at least one LED (11).

15. A microcrystalline wax housing perfume diffusing device of claim 13, wherein said perfumed volatile material (3) is a scent.

16. A microcrystalline wax housing perfume diffusing device of claim 13, wherein said perfumed volatile material (3) is a natural dried flower.

17. A microcrystalline wax housing perfume diffusing device of claim 13, wherein said perfumed volatile material (3) is a concentrated scent wax particle.

18. A microcrystalline wax housing perfume diffusing device of claim 13, wherein an electronic component (1) is installed inside the microcrystalline wax housing (2), the electronic component (1) has an LED (11), a battery compartment (12) and a power switch (13).

19. A microcrystalline wax housing perfume diffusing device of claim 13 wherein said microcrystalline wax housing is capable of withstanding high-temperature of 72˜80° C.

20. A microcrystalline wax housing perfume diffusing device of claim 13, wherein the microcrystalline wax housing (2) of the present invention is more shatterproof when falling from the height of about 80˜120 cm.
